(19) United States
(12) Patent Application Publication
(10) Pub. No.: US 2020/0129472 A1
(43) Pub. Date: Apr. 30, 2020

(54) COMPOSITIONS AND METHODS FOR THE TREATMENT OF PERIPHERAL ARTERY DISEASE

(71) Applicant: BOARD OF REGENTS OF THE UNIVERSITY OF NEBRASKA, Lincoln, NE (US)

(72) Inventors: Hanjun WANG, Omaha, NE (US); Irving ZUCKER, Omaha, NE (US); Iraklis PIPINOS, Omaha, NE (US); Steven LISCO, Omaha, NE (US); Ting LI, Omaha, NE (US)

(21) Appl. No.: 16/483,548

(22) PCT Filed: Feb. 9, 2018
(86) PCT No.: PCT/US2018/017594

§ 371 Date: Aug. 5, 2019

Related U.S. Application Data
(60) Provisional application No. 62/456,763, filed on Feb. 9, 2017.

(51) Int. Cl. A61K 31/357; A61K 9/00; A61P 9/00; A61K 45/06

(52) U.S. Cl. CPC A61K 31/357; A61K 9/0019; A61K 45/06; A61P 9/00; A61K 9/0085

(57) ABSTRACT

Compositions and methods for the treatment of peripheral artery disease and the symptoms thereof are provided.

This application claims priority under 35 U.S.C. § 119(e) to U.S. Provisional Patent Application No. 62/456,763, filed on Feb. 9, 2017. The foregoing application is incorporated by reference herein.

### FIELD OF THE INVENTION

This invention relates generally to the field of atherosclerosis. Specifically, the invention provides compositions and methods for the treatment of peripheral artery disease.

### BACKGROUND OF THE INVENTION

Peripheral artery disease (PAD) is a manifestation of systemic atherosclerosis affecting around 8 to 12 million people in the United States. Symptoms of PAD include claudication, resting pain, and tissue loss which are all consequences of skeletal myopathy and skeletal muscle sensory dysfunction. The exercise pressor reflex (EPR) is a neural reflex originating in skeletal muscle that contributes to the regulation of the cardiovascular and respiratory systems during physical activity. The sensory arm of this reflex is composed of both metabolically sensitive (group IV) and mechanically sensitive (group III) nerves. Evidence from human and animal studies has demonstrated that increases in heart rate (HR), arterial pressure (AP) and sympathetic nerve activity in response to activation of this reflex are enhanced in PAD patients and animals, indicating that an exaggerated EPR exists in the PAD state. The exaggerated EPR can cause a potent vasoconstriction and limit blood flow to exercising muscle, which can contribute to the symptom of exercise intolerance in the PAD patients. Improved therapeutics for treating PAD and the symptoms associated therewith are needed.

### SUMMARY OF THE INVENTION

In accordance with the instant invention, methods of inhibiting, treating, and/or preventing peripheral artery disease or symptoms associated therewith are provided. In a particular embodiment, the methods inhibit, treat, and/or prevent claudication associated with peripheral artery disease. In a particular embodiment, the method improves the exercise performance of the subject. Compositions for use in these methods are also provided. In a particular embodiment, the subject being treated by the methods of the instant invention has an ankle-brachial index ratio less than 0.9, less than 0.8, less than 0.7, less than 0.6, or less than 0.5.

In a particular embodiment, the methods comprise administering an antagonist or an agonist of transient receptor potential cation channel subfamily V member 1 (TRPV1), particularly a TRPV1 agonist such as resiniferatoxin. In a particular embodiment, the methods comprise administering the TRPV1 agonist (or antagonist) by intrathecal administration, epidural administration, or intraganglionic administration. In a particular embodiment, the agonist (or antagonist) of TRPV1 is administered via an epidural injection within the lumbar dorsal root ganglion, particularly at one or more of the lumbar regions L1-L5, particularly at L4 and/or L5.

The methods of the instant invention may further comprise administering at least one other therapeutic for the treatment of peripheral artery disease and/or a symptom thereof. The methods of the instant invention may further comprise diagnosing peripheral artery disease in the subject prior to administration of the therapy.

### BRIEF DESCRIPTIONS OF THE DRAWING

FIGS. 1A-1D provide graphs showing that resiniferatoxin (RTX) administration reduces exercise limitations in PAD rats. FIG. 1A provides the running distance over time by healthy sham-treated rats or PAD rats mock treated (control) or treated with RTX. FIG. 1B provides the maximum running speed over time by healthy sham-treated rats or PAD rats mock treated (control) or treated with RTX. FIG. 1C provides the time run at 37 m/min by healthy sham-treated rats or PAD rats mock treated (control) or treated with RTX. FIG. 1D provides the time running at percent maximum intensity by healthy sham-treated rats or PAD rats mock treated (control) or treated with RTX. Mean ±standard error (SE). n=7 in sham and PAD+control rats. n=6 in PAD+RTX rats. *P<0.05 vs. sham. †P<0.05 vs. PAD+control.

FIGS. 2A-2D provide graphs showing that resiniferatoxin (RTX) administration reduces exercise limitations in PAD rats when administered 4 weeks post femoral artery occlusion. FIG. 2A provides the maximum speed by PAD rats before and after mock treatment (control) or treatment with RTX. FIG. 2B provides the running distance by PAD rats before and after mock treatment (control) or treatment with RTX. FIG. 2C provides the time run at 37 m/min by PAD rats before and after mock treatment (control). FIG. 2D provides the time run at 37 m/min by PAD rats before and after or treated with RTX. Mean ±SE. n=6 in PAD+control. n=8 in PAD+RTX. *P<0.05 vs. before.

### DETAILED DESCRIPTION OF THE INVENTION

Atherosclerosis is the accumulation of plaques on vascular walls. The presence of atherosclerotic plaques can severely diminish vascular flow to target organs, leading to morbidity and mortality. Peripheral artery disease (PAD) occurs when atherosclerotic plaques arise in peripheral arteries, such as in the limbs, particularly the legs. Patients with PAD are at increased risk for decreased mobility, ulcers, gangrene, myocardial infarction, cerebrovascular attack, aortic aneurym rupture, and vascular death (Criqui, et al, (1997) Vasc. Med., 2:221-6; Meijer, et al. (1998) Arterioscler. Thromb. Vase Biol., 18:185-92).

One of the symptoms of PAD is claudication. Claudication is pain and/or cramping caused by too little blood flow to a subject's limbs, particularly the legs. The pain and/or cramping may occur not only during light or strenuous exercise or physical activity, but also when the subject is at rest. Contracting muscle during dynamic exercise releases many metabolites, most of which cause potent vasodilation and increase blood flow and oxygen delivery to the contracting muscles. In contrast, the exercise pressor reflex (EPR) causes increased sympathetic outflow to the muscles during exercise which limits blood flow and oxygen delivery to the contracting muscles. Without being bound by theory, the endothelium dysfunction in PAD subjects largely blunts the metabolites-induced vasodilator effect. Moreover, the ischemic-induced afferent sensitization in PAD subjects increases or exaggerates the EPR-induced vasoconstriction. Thus, the net blood flow response during exercise in PAD subjects shifts to the vasoconstriction direction, thereby causing the claudication. Herein, it is demonstrated that the inhibition, desensitization, and/or ablation of skeletal muscle afferent nerves (e.g., by administration of resiniferatoxin) interrupts EPR signaling transduction in PAD subjects, thereby improving blood flow and exercise performance while reducing claudication.

The instant invention encompasses methods of inhibiting, treating, and/or preventing peripheral artery disease and/or the symptoms associated therewith. In a particular embodiment, the methods inhibit, treat, and/or prevent claudication associated with peripheral artery disease. In a particular embodiment, the methods inhibit, treat, and/or prevent muscle inflammation and/or fibrosis. In a particular embodiment, the methods improve exercise performance (e.g., ability of a subject to perform a specified exercise) and/or improve hemodynamic dysfunction in the subject (e.g., as compared to the subject before therapy). The methods may comprise administering at least one agent or compound which inhibits, desensitizes, and/or ablates skeletal muscle afferent nerves (e.g., TRPV1 positive skeletal muscle afferent nerves). In a particular embodiment, the methods of the instant invention comprise administering at least one agonist or antagonist of transient receptor potential cation channel subfamily V member 1 (TRPV1; also known as capsaicin receptor, Osm-9-like TRP channel 1 (OTRPC1), and vanilloid receptor 1; see, e.g., GenBank Gene ID: 7442), particularly a TRPV1 agonist, to a subject. In a particular embodiment, the agonist or antagonist is a small molecule.

Without being bound by theory, TRPV1 agonists such as capsaicin will cause the excessive calcium influx into the TRPV1-positive sensory neurons and induce cell death. Low doses of strong agonists such as RTX (e.g., <6 μg/ml) will also ablate the TRPV1-positive sensory neurons and, therefore, diminish the exercise pressor reflex. On the other hand, TRPV1 antagonists can also at least partially inhibit the TRPV1-positive sensory afferent function and suppress the exercise pressor reflex in the PAD subjects. Therefore, either TRPV1 agonists (e.g., by destroying neurons) or TRPV1 antagonists (e.g., by inhibiting the sensory neurons function) can abolish or inhibit the exercise pressor reflex in the PAD state.

Examples of TRPV1 agonists include, without limitation, capsaicin (including capsaicin cream (e.g., Qutenza®)), N-oleoyldopamine (OLDA), olvanil (N-9-Z-octadecenoyl-vanillamide), and resiniferatoxin (RTX; [(1R,6R,13R,15R,17R)-13-benzyl-6-hydroxy-4,17-dimethyl-5-oxo-15-(prop-1-en-2-yl)-12,14,18-trioxapentacyclo[11.4.1.0^{1,10}.0^{2,6}.0^{11,15}]octadeca-3,8-dien-8-yl]methyl 2-(4-hydroxy-3-methoxyphenyl)acetate). In a particular embodiment, the TRPV1 agonist is RTX.

Examples of TRPV1 antagonists include, without limitation:

XEN-0501 (XEN-D0501) (Belvisi et al., Am. J. Respir. Crit. Care Med. (2017) 196(10):1255-1263; Round et al., Br. J. Clin. Pharmacol. (2011) 72(6):921-31),

GRC-6211 (Charrua et al., J. Urol. (2009) 181(1):379-86),

JYL-1421 (1-[(4-tert-butylphenyl)methyl]-3-[[3-fluoro-4-(methanesulfonamido) phenyl]methyl]thiourea),

capsazepine (N-[2-(4-Chlorophenyl)ethyl]-1,3,4,5-tetrahydro-7,8-dihydroxy-2H-2-benzazepine-2-carbothioamide),

SB-705498 (N-(2-bromophenyl)-N′-[((R)-1-(5-trifluoromethyl-2-pyridyl) pyrrolidine-3-yl)]urea),

SB-452533 (N-(2-bromophenyl)-N′-[2-[ethyl(3-methylphenyl)amino]ethyl]urea),

SB-366791 (N-(3-Methoxyphenyl)-4-chlorocinnamide),

SB-782443 (6-(4-fluorophenyl)-2-methyl-N-(2-methylbenzothiazol-5-yl)nicotinamide),

A-425619 (1-isoquinolin-5-yl-3-(4-trifluoromethyl-benzyl)-urea),

A-784168 (3,6-Dihydro-3′-(trifluoromethyl)-N-[4-[(trifluoromethyl)sulfonyl]phenyl]-[1(2H),2′-bipyridine]-4-carboxamide),

A-795614 ((R)-1-(1H-indazol-4-yl)-3-(5-(piperidin-1-yl)-2,3-dihydro-1H-inden-1-yl)urea),

ABT-102 ((R)-1-(5-tert-butyl-2,3-dihydro-1H-inden-l-yl)-3-(1H-indazol-4-yl)urea),

AMG9810 (2E-N-(2,3-Dihydro-1,4-benzodioxin-6-yl)-3-[4-(1,1-dimethylethyl) phenyl]-2-Propenamide),

AMG0347 (N-(7-hydroxy-5,6,7,8-tetrahydronaphthalen-1-yl)-3-[2-piperidin-1-yl-6-(trifluoromethyl)pyridin-3-yl]prop-2-enamide),

AMG517 (N-(4-[6-(4-trifluoromethyl-phenyl)-pyrimidin-4-yloxy]-benzothiazol-2-yl)-acetamide I),

AMG8163 (tert-butyl-2-(6-([2-(acetylamino)-1,3-benzothiazol-4-yl]oxy)pyrimidin-4-yl)-5-(trifluoromethyl)phenylcarbamate),

iodo-resiniferatoxin (I-RTX),

N-(4-tertiarybutylphenyl)-4-(3-chloropyridin-2-yl)tetrahydropyrazine-1(2H)-carbox-amide (BCTC),

ND68243, and

AZD1386 (5′-chloro-7′-methyl-1′-[[3-(trifluoromethyl)phenyl]methyl]spiro[imidazolidine-5,3′-indole]-2,2′,4-trione).

The methods of the instant invention may further comprise the administration (sequentially (e.g., before and/or after) and/or simultaneously) of at least one other therapeutic for the treatment of peripheral artery disease and/or a symptom thereof. For example, the methods may further comprise administering medications known to treat atherosclerosis and/or prevent heart attacks or strokes in patients with atherosclerosis. Examples of therapeutic agents that may be administered in the instant methods include, without limitation: anti-platelet agents, cholesterol-lowering drugs (e.g., statins, selective cholesterol absorption inhibitors, and resins), high blood pressure medication (e.g, diuretics, angiotensin converting enzyme (ACE) inhibitors, beta-blockers, angiotensin II receptor blockers, calcium channel blockers, alpha blockers, alpha-2 receptor agonists, central agonists, peripheral adrenergic inhibitors, and vasodilators), and antiarrythmics.

The methods of the instant invention may further comprise diagnosing peripheral artery disease in the subject prior to administration of the therapeutic agents of the instant invention. For example, PAD may be diagnosed by the ankle-brachial index. Briefly, the blood pressure is taken at an upper extremity (e.g., the arm) and at a lower extremity (e.g., the foot or ankle) and then the ratio of the systolic pressure in the lower extremity to that in the upper extremity is calculated. If the ratio is less than 0.90, PAD is diagnosed. Generally, the lower the ratio, the more severe the disease. For example, severe arterial narrowing is diagnosed when the ratio is less than about 0.50. In a particular embodiment, the subject being treated by the methods of the instant invention has an ankle-brachial index ratio less than 0.9, less than 0.8, less than 0.7, less than 0.6, or less than 0.5.

The instant invention also encompasses compositions, particularly for inhibiting, treating, and/or preventing peripheral artery disease and/or the symptoms (e.g., claudication) associated therewith. The compositions comprise i) at least one at least one agent or compound which inhibits, desensitizes, and/or ablates skeletal muscle afferent nerves (e.g., TRPV1 positive skeletal muscle afferent nerves), particularly at least one TRPV1 agonist or antagonist, more particularly at least one TRPV1 agonist such as RTX, and ii) at least one pharmaceutically acceptable carrier. In a particular embodiment, the composition further comprises at least one other therapeutic agent for the treatment of the peripheral artery disease and/or a symptom thereof, as described above.

The therapeutic agents (e.g., a TRPV1 agonist) of the present invention can be administered by any suitable route, for example, by injection (e.g., for local, direct, or systemic administration), oral, pulmonary, topical, nasal or other modes of administration. The therapeutic agents may be contained within a composition with at least one pharmaceutically acceptable carrier. The composition may be administered by any suitable means including, for example: by injection or by parenteral, intramuscular, intravenous, intraarterial, intraperitoneal, subcutaneous, oral, topical, inhalatory, transdermal, intrapulmonary, intraareterial, intrarectal, intramuscular, intranasal, intrathecal, epidural, intraganglionic, and intra-spinal administration. In a particular embodiment, the composition is administered by intrathecal administration, epidural administration, or intraganglionic administration. In a particular embodiment, the composition is administered via an epidural injection within the lumbar dorsal root ganglions (DRGs). In a particular embodiment, the epidural injection is made at one or more of the lumbar regions L1-L5. In a particular embodiment, the epidural injection is at L4 and/or L5.

In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), antioxidants (e.g., ascorbic acid, sodium metabisulfite), preservatives (e.g., Thimersol, benzyl alcohol) and bulking substances (e.g., lactose, mannitol). Common carriers include, without limitation, water, oil, buffered saline, ethanol, polyol (for example, glycerol, propylene glycol, liquid polyethylene glycol and the like), dimethyl sulfoxide (DMSO), detergents, suspending agents, glucose, lactose, gum acacia, gelatin, mannitol, starch paste, magnesium trisilicate, talc, corn starch, keratin, colloidal silica, potato starch, urea, medium chain length triglycerides, dextrans, other carriers suitable for use in manufacturing preparations, in solid, semisolid, or liquid form, and suitable mixtures thereof. In addition excipients and auxiliary, stabilizing, preserving, thickening, flavoring, and coloring agents may be included in the compositions. The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can be prepared, for example, in liquid form, or can be in dried powder form (e.g., lyophilized for later reconstitution).

The therapeutic agents described herein will generally be administered to a subject/patient as a pharmaceutical preparation. The term “patient” as used herein refers to human or animal subjects. The compositions of the instant invention may be employed therapeutically or prophylactically, under the guidance of a physician. The compositions comprising the agent of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated.

The dose and dosage regimen of the therapeutic agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular condition may be determined by dosage concentration curve calculations, as known in the art.

The pharmaceutical preparation comprising the therapeutic agent may be administered at appropriate intervals until the pathological symptoms are reduced or alleviated, after which the dosage may be reduced to a maintenance level. The appropriate interval in a particular case would normally depend on the condition of the patient.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### DEFINITIONS

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition or symptom resulting in a decrease in the probability that the subject will develop the condition or symptom.

A “therapeutically effective amount” of a compound or a pharmaceutical composition refers to an amount effective to prevent, inhibit, treat, or lessen the symptoms of a particular disorder or disease. The treatment of peripheral artery disease herein may refer to curing, relieving, and/or preventing peripheral artery disease, the symptom(s) of it, or the predisposition towards it.

As used herein, the term “subject” refers to an animal, particularly a mammal, particularly a human.

As used herein, “diagnose” refers to detecting and identifying a disease or disorder in a subject. The term may also encompass assessing or evaluating the disease or disorder status (severity, progression, regression, stabilization, response to treatment, etc.) in a patient known to have the disease or disorder.

As used herein, the term “prognosis” refers to providing information regarding the impact of the presence of a disease or disorder (e.g., as determined by the diagnostic methods of the present invention) on a subject's future health (e.g., expected morbidity or mortality). In other words, the term “prognosis” refers to providing a prediction of the probable course and outcome of a disease/disorder or the likelihood of recovery from the disease/disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “exercise performance” refers to physical acts or exertion which typically dependent on skeletal muscle contraction. Examples of exercise performance include, without limitation, running (e.g., speed and/or endurance), walking (e.g., speed and/or endurance), swimming (e.g., speed and/or endurance), and lifting (e.g., strength and/or endurance).

The following example describes illustrative methods of practicing the instant invention and is not intended to limit the scope of the invention in any way.

### EXAMPLE

### Methods

### PAD Rat Model

Catheter-based femoral artery occlusion was created by placing a modified PE50 catheter (its cavity filled with solid agarose) between common iliac artery and left femoral artery to interrupt hindlimb blood supply. The whole procedure is similar to the telemetry implant surgery. Generally, rats were anesthetized using a 2%-3% isoflurane:oxygen mixture. A warm water blanket was used to provide intra-operative heat support to prevent hypothermia. Rats were placed in the prone position and the surgical site (i.e., the femoral area) was cleared of hair, prepared with iodine or chlorhexidine, and the femoral artery was exposed. After a small incision was made, a modified PE50 catheter was placed into the femoral artery and advanced about 3.5 cm to the iliac artery at the aortic bifurcation. The distal end of the femoral artery was ligated with 4-0 Dexon suture. The skin was closed with external interrupted 4-0 or 3-0 prolene suture, which were removed in 10-14 days after the surgery.

### Exercise Intolerance Test

Exercise testing for determination of intolerance time was performed in a motorized treadmill equipped with an electrical grid at the end of the lane. The animals were initially acclimated to the treadmill environment at a low speed (13 m/min, 0% grade) for 6 minutes. Then, data was recorded of the exercise duration and distance at the speed of the treadmill of 13 m/minute (0% grade) and increased of 3 m/minute each 2 minutes until exhaustion. Exhaustion was defined operationally as the time at which the rat was unable, or refused, to maintain its running speed for more than 15 seconds despite encouragement by mild electrical stimulation. The maximal speed and running distance were recorded when exhaustion occurred. Two methods were used to measure the exercise intolerance time in sham, PAD and PAD+RTX rats. With the first method, individual rats were run with 90%, 75%, 50% and 25% of its own maximal speed. The exercise intolerance time corresponding to individual running intensity was recorded and compared among groups. With the second method, a fixed running speed (37 m/minute) was used to run all rats among groups to get their exercise intolerance time.

### Epidural Delivery of RTX

To deplete skeletal muscle afferent neuron soma (L4-L5 DRGs), rats were anesthetized using 2%-3% isoflurane:oxygen mixture. Rats were placed in the prone position and the surgical site was cleared of hair, prepared with iodine or chlorhexidine. A small midline incision was made in the region of the T13-L1 thoracic vertebrae. Following dissection of the superficial muscles, a small hole (approximately 3 mm*3 mm) was made in the left side of T13 vertebral (ipsilateral). A polyethylene catheter (PE-10) was inserted into the subarachnoid space via left hole and gently advanced about 4.0 cm to the left L5 level in which the first injection (6 μg/ml, 10 μl) was made at a very slow speed to minimize the diffusion of RTX. Then the catheter was pulled back to left L4 to perform another injection (10 μl/each). Then the catheter was held for 15 minutes and then withdrawn. After that, silicon gels were used to seal the hole in the T13 vertebral. The skin overlying the muscle was closured by 3-0 polypropylene suture. Simple interrupted sutures were used to close the skin. Skin suture were removed 10-14 days after surgery. Betadine was applied to the wound and the rats were allowed to recover from the anesthesia.

### Results

The therapeutic efficacy of resiniferatoxin was tested in a in a rat model of peripheral arterial disease. As explained above, resiniferatoxin was administered to PAD rats by lumbar epidural administration at the time of femoral arterial occlusion. The resiniferatoxin (6 μg/ml, 10 μl/per ganglia) was injected between the ipsilateral L4 and L5 vertebrae into the epidural space at a concentration of 10 μl per ganglion. Healthy rats with sham treatment were also used as a positive control.

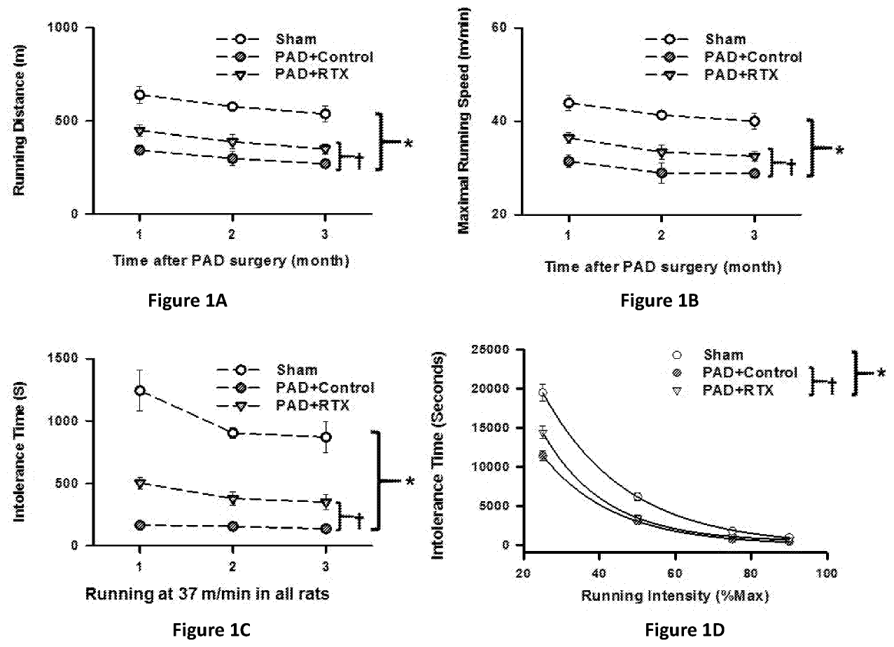

As seen in FIG. 1, resiniferatoxin administration reduces the exercise limitations associated with PAD. Indeed, in PAD rats, resiniferatoxin administration resulted in increased running distances, increased maximal running speed, an increased ability to run at 37 m/minute, and an increased ability to run for longer distances at a higher rate. These statistically significant improvements lasted for months after PAD surgery.

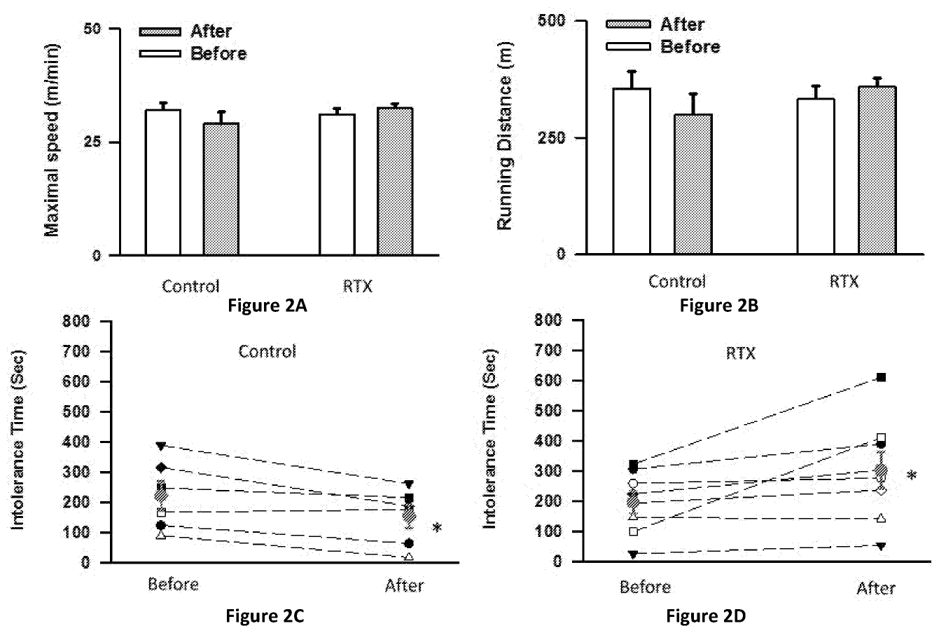

The ability of resiniferatoxin administration to reduce the exercise limitations associated with PAD when administered 4 weeks after femoral arterial occlusion was also tested. As seen in FIG. 2, resiniferatoxin administration reduced the exercise limitations associated with PAD even when administered after the onset of PAD.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

Several publications and patent documents are cited in the foregoing specification in order to more fully describe the state of the art to which this invention pertains. The disclosure of each of these citations is incorporated by reference herein.

1. A method for inhibiting or treating peripheral artery disease in a subject, said method comprising administering an agonist of transient receptor potential cation channel subfamily V member 1 (TRPV1) to said subject.

2. The method of claim 1, wherein said agonist of TRPV1 is selected from the group consisting of capsaicin, N-oleoyldopamine (OLDA), olvanil (N-9-Z-octadecenoyl-vanillamide), and resiniferatoxin

3. The method of claim 2, wherein said agonist of TRPV1 is resiniferatoxin.

4. The method of claim 1, wherein said method inhibits or treats claudication associated with said peripheral artery disease.

5. The method of claim 1, wherein said method improves the exercise performance of the subject.

6. The method of claim 1, wherein said agonist of TRPV1 is administered by intrathecal administration, epidural administration, or intraganglionic administration.

7. The method of claim 6, wherein said agonist of TRPV1 is administered via an epidural injection within the lumbar dorsal root ganglion.

8. The method of claim 6, wherein said agonist of TRPV1 is administered via an epidural injection at one or more of the lumbar regions L1-L5.

9. The method of claim 6, wherein said agonist of TRPV1 is administered via an epidural injection at L4 and/or L5.

10. The method of claim 1, further comprising administration of at least one other therapeutic for the treatment of peripheral artery disease and/or a symptom thereof.

11. The method of claim 10, wherein said other therapeutic is selected from the group consisting of anti-platelet agents, cholesterol-lowering drugs, high blood pressure medication, and antiarrythmics.

12. The method of claim 1, further comprising diagnosing peripheral artery disease in the subject prior to administration of said agonist of TRPV1.
